(19) United States
(12) Patent Application Publication
(10) Pub. No.: US 2025/0276135 A1
(43) Pub. Date: Sep. 4, 2025

(54) ATOMIZER

(71) Applicant: SUZHOU BOYUNXING TECHNOLOGY CO., LTD., SUZHOU (CN)

(72) Inventor: Feng WANG, SUZHOU (CN)

(73) Assignee: SUZHOU BOYUNXING TECHNOLOGY CO., LTD., SUZHOU (CN)

(21) Appl. No.: 19/210,827

(22) Filed: May 16, 2025

Related U.S. Application Data
(63) Continuation of application No. PCT/CN2023/132418, filed on Nov. 17, 2023.

(30) Foreign Application Priority Data
Nov. 24, 2022 (CN) 202211485076.9

(51) Int. Cl. A61M 11/00

(52) U.S. Cl. CPC A61M 11/006; A61M 2205/276

(57) ABSTRACT

An atomizer is provided. The atomizer includes: a delivery tube holder; a mouthpiece, which has a first end configured to be placed at a user's mouth, and includes a mouthpiece body and a first locking structure arranged on the mouthpiece body; an actuating member, which is located at a second end of the mouthpiece opposite to the first end, and includes an actuating member body and a second locking structure arranged on the actuating member body, the actuating member can pivot relative to the mouthpiece to drive the delivery tube holder to pivot; the actuating member has a locked position, where the first locking structure engages with the second locking structure to block the actuating member from pivoting relative to the mouthpiece, and an unlocked position, where the first locking structure does not engage with the second locking structure such that the actuating member can pivot relative to the mouthpiece.

### CROSS REFERENCE TO RELATED APPLICATIONS

The present application is a continuation of the international application PCT/CN2023/132418, filed on Nov. 17, 2023 and entitled “NEBULIZER”, and the international application claims the priority to Chinese Patent Application No. 202211485076.9 filed on Nov. 24, 2022, which is incorporated herein by reference in its entirety.

### TECHNICAL FIELD

The disclosure relates to the technical field of medical instruments. Specifically, the disclosure relates to an atomizer which may be used, for example, for atomizing and/or spraying a medicinal liquid.

### BACKGROUND

In the related art, a container in an atomizer (or referred to as a sprayer) contains a liquid to be atomized or sprayed, and during a movement stroke of the container relative to a spraying assembly (such as a pump), the liquid in the container is atomized, and the atomized liquid is sprayed from a nozzle of the spraying assembly. However, the triggering of such an atomizer is usually not stable enough, and the atomizer may be automatically triggered when a user exerts too much force. In addition, when the atomizer is subjected to vibration or shaking, such as accidental slipping, internal components may automatically move undesirably, causing the atomizer to be automatically triggered without human intervention.

This accidental automatic triggering of the atomizer is undesired, which not only leads to an ineffective waste of the liquid, but may also lead to leakage, or even damage or failure of the atomizer when a user is not using the atomizer.

### SUMMARY OF THE INVENTION

The disclosure provides an atomizer.

According to the disclosure, an atomizer is provided. The atomizer includes: a delivery tube holder; a mouthpiece, which has a first end configured to be placed at the mouth of a user, and includes a mouthpiece body and a first locking structure arranged on the mouthpiece body; and an actuating member, which is located at a second end of the mouthpiece opposite to the first end, and includes an actuating member body and a second locking structure arranged on the actuating member body, where the actuating member is capable of being pivoted relative to the mouthpiece to drive the delivery tube holder to pivot; and where the actuating member has a locked position, in which the first locking structure engages with the second locking structure to block the actuating member from pivoting relative to the mouthpiece, and an unlocked position, in which the first locking structure does not engage with the second locking structure such that the actuating member is capable of pivoting relative to the mouthpiece.

According to some embodiments, the first locking structure includes a locking slider, and the second locking structure includes a slot for accommodating the locking slider.

According to some embodiments, the locking slider is capable of moving relative to the mouthpiece body in an axial direction of the mouthpiece, and the slot extends in the axial direction from an end surface of the actuating member body facing the mouthpiece.

According to some embodiments, in the locked position, a portion of the locking slider in the axial direction is inserted into the slot to block the actuating member from pivoting relative to the mouthpiece; and in the unlocked position, the locking slider abuts on the end surface of the actuating member body facing the mouthpiece.

According to some embodiments, the mouthpiece body includes a recess for accommodating the locking slider, and the first locking structure further includes an elastic member, one end of the elastic member being fixed to an inner wall of the recess, and the other end of the elastic member being fixed to the locking slider.

According to some embodiments, in the unlocked position, the elastic member is in a compressed state; and during the movement of the actuating member from the unlocked position to the locked position, the clastic member returns to enable the locking slider to enter the slot.

According to some embodiments, a circumferential surface of the delivery tube holder is provided with a protrusion extending into the slot, and in the locked position, the locking slider abuts on the protrusion, such that during the movement of the delivery tube holder toward the mouthpiece, the protrusion pushes the locking slider to return to the mouthpiece body.

According to some embodiments, in the unlocked position, the protrusion comes into contact with the slot, such that the actuating member is capable of pushing the delivery tube holder to pivot via the protrusion.

According to some embodiments, a side wall of the slot is provided with an inclined surface inclined from the side wall toward the end surface.

According to some embodiments, when the atomizer is in an initial state, the actuating member is in the unlocked position; and when the atomizer is in a pre-triggering state, the actuating member is in the locked position.

According to some embodiments, the atomizer further includes a switch button, where in the locked position, the switch button is capable of being pressed to drive the delivery tube holder to pivot.

According to some embodiments, a first inclined surface is provided at an end portion of the switch button facing the delivery tube holder, and the first inclined surface is configured to be inclined from the end portion away from the delivery tube holder; and an opening is provided in a circumferential wall of the delivery tube holder, and the opening is configured to accommodate the end portion.

According to some embodiments, in the locked position, the end portion faces a first side wall of the opening, and during a process of the switch button being pressed, the first inclined surface is capable of coming into contact with the first side wall to push the delivery tube holder to pivot.

According to some embodiments, in the unlocked position, the end portion faces the circumferential wall of the delivery tube holder.

According to some embodiments, a second side wall of the opening opposite to the first side wall is provided with a second inclined surface inclined from the second side wall toward the switch button; and when the atomizer is in a triggered state, the actuating member is pivoted to cause the second inclined surface to come into contact with the end portion, so as to enable the switch button to move away from the delivery tube holder.

According to some embodiments, the end portion of the switch button is further provided with a third inclined surface opposite to the first inclined surface, and the third inclined surface is configured to be inclined from the end portion away from the delivery tube holder.

According to some embodiments, where a first spiral portion is provided on an inner wall of the mouthpiece, and a second spiral portion is provided on an inner wall of the delivery tube holder facing the inner wall of the mouthpiece; and where the first spiral portion is capable of coming into contact with the second spiral portion, so as to enable the delivery tube holder to move in the axial direction while pivoting.

According to some embodiments, in the pre-triggering state, the highest point of the first spiral portion in the axial direction coincides with the highest point of the second spiral portion in the axial direction.

According to some embodiments, a width of the first spiral portion in a radial direction of the delivery tube holder is different from a width of the second spiral portion in the radial direction.

### BRIEF DESCRIPTION OF THE DRAWINGS

To describe technical solutions in embodiments of the disclosure or in the prior art more clearly, accompanying drawings required for description of the embodiments or the prior art will be briefly introduced below. Apparently, the accompanying drawings in the following description show some embodiments of the disclosure merely, and those of ordinary skill in the art can derive other drawings from structures shown in these accompanying drawings without any creative effort.

In the figures:

FIG. 1 shows a front view of an atomizer according to some embodiments of the disclosure;

FIG. 2 shows a cross-sectional view of the atomizer in FIG. 1 taken along R-R;

FIG. 3 shows an exploded view of the atomizer in FIG. 1;

FIG. 4 shows a perspective view of some components of the atomizer in FIG. 1;

FIGS. 5A to 5C show schematic diagrams of the atomizer in FIG. 1 in a pre-triggering state from different perspectives, FIG. 5A showing a cross-sectional view of the atomizer in FIG. 1 in the pre-triggering state taken along N-N, FIG. 5B showing a schematic diagram of some components of the atomizer in the pre-triggering state, and FIG. 5C showing a cross-sectional view of some components in FIG. 5B taken along D-D;

FIGS. 6A to 6C show schematic diagrams of an atomizer in FIG. 1 in a triggered state from different perspectives, FIG. 6A showing a cross-sectional view of the atomizer in FIG. 1 in the triggered state taken along N-N, FIG. 6B showing a schematic diagram of some components of the atomizer in the triggered state, and FIG. 6C showing a cross-sectional view of some components in FIG. 6B taken along E-E;

FIG. 7A shows a schematic diagram of some components of the atomizer in FIG. 1 when an actuating member is in an unlocked position;

FIG. 7B shows a cross-sectional view of a triggering assembly in FIG. 7A taken along K- K;

FIG. 8 shows a schematic diagram of the actuating member of the atomizer in FIG. 1;

FIG. 9 shows a schematic diagram of a locking slider of the atomizer in FIG. 1;

FIG. 10 shows a schematic diagram of a mouthpiece of the atomizer in FIG. 1;

FIG. 11 shows a schematic diagram of a delivery tube holder of the atomizer in FIG. 1; and

FIG. 12 shows a schematic diagram of a switch button of the atomizer in FIG. 1.

In the figures, the same reference signs refer to the same or similar features.

The implementation of the objective, functional characteristics, and advantages of the disclosure will be further described with reference to the accompanying drawings in combination with embodiments.

### DETAILED DESCRIPTION

In the scope of the disclosure, an “atomizer” (also referred to as a “sprayer”) refers to a device for atomizing a liquid. Generally, the atomizer is configured to atomize a fluid (e.g., a medicinal liquid or the like) and spray the atomized fluid to some parts of a user (such as a patient) to be treated. Since the medicinal liquid is loaded in the atomizer, the stability of the atomizer is particularly important.

In the related art, the atomizer may be operated by mechanical means, such as a push switch or a rotary switch. However, the locking of the atomizer by the switches may be unreliable. For example, when the atomizer is in a pre-triggering state, if a housing of the atomizer continues to rotate, automatic triggering may occur. It should be understood that this undesired atomization trigger is ineffective because the medicinal liquid does not reach the site to be treated in this case.

In the disclosure, locking structures are provided on the mouthpiece and the actuating member, respectively, so as to lock or release the actuating member relative to the mouthpiece, which can effectively prevent the automatic triggering of the atomizer. For example, when a user rotates the housing to actuate the atomizer, it is possible to prevent undesired accidental triggering of the atomizer due to the user rotating the housing too much. For another example, when the atomizer slips off accidentally, the moving parts (such as the actuating member) in the atomizer can be securely locked, so that the atomizer is stably located in a non-triggered position. It is also advantageous that in the locked position, the mouthpiece as a fixing member can lock the movement of the delivery tube holder. Such locking is efficient and secure, and other unnecessary locking parts can be omitted, such that the atomizer has a more compact structure and is cost-saving. In this way, it is possible to prevent automatic triggering of the atomizer simply, effectively and reliably.

In the scope of the disclosure, the “locked position” of the actuating member refers to a position in which the actuating member is locked, that is, the atomizer cannot be triggered without the action of other components. In other words, when the actuating member is in the “locked position”, the atomizer can perform an atomization or spraying operation only manually by means of other components.

In the scope of the disclosure, the “unlocked position” of the actuating member refers to a position in which the actuating member is not locked, that is, a position in which the atomizer can be operated by means of the actuating member. In other words, when the actuating member is in the “unlocked position”, a user can rotate the actuating member to drive the delivery tube holder to perform corresponding rotation and axial movement at the same time.

In the scope of the disclosure, the “initial state” of the atomizer refers to a state in which no operation is performed on the atomizer, the “pre-triggering state” refers to a critical state of the atomizer before being triggered, and the “triggered state” refers to a state in which the atomizer has been triggered, that is, the spaying has just been completed and no other operations are performed. Specifically, the initial state, the pre-triggering state and the triggered state of the atomizer may be switched by, for example, the following process. From the initial state, the actuating member is rotated to enable the delivery tube holder to rotate downward to the lowest position relative to the mouthpiece, and at this time the atomizer enters the pre-triggering state. In the pre-triggering state, the delivery tube holder continues to rotate, such that the delivery tube holder can move toward a spraying assembly under the action of a bottom spring of the delivery tube holder, at this time the medicinal liquid in a container connected to the delivery tube holder is atomized and sprayed, and thus the atomizer completes the spaying operation and enters the triggered state.

An atomizer according to the disclosure will be described in detail below with reference to the embodiments shown in the drawings.

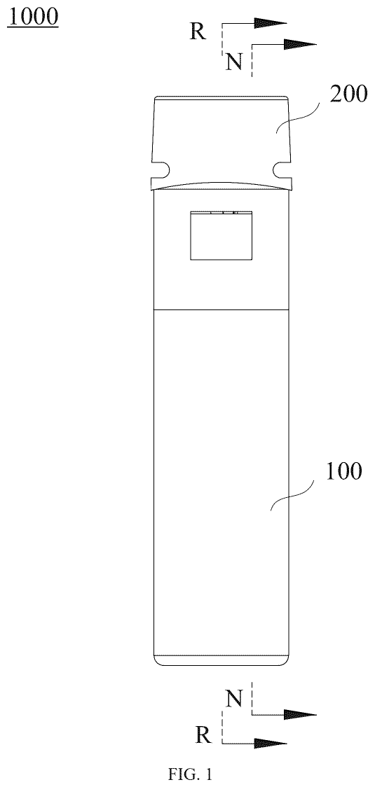

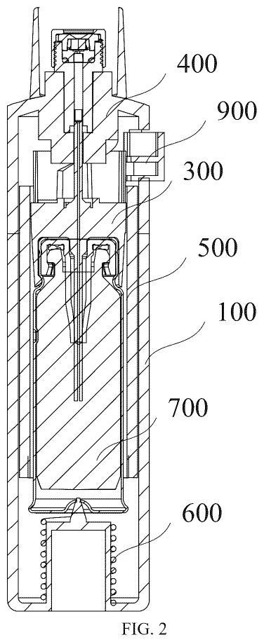

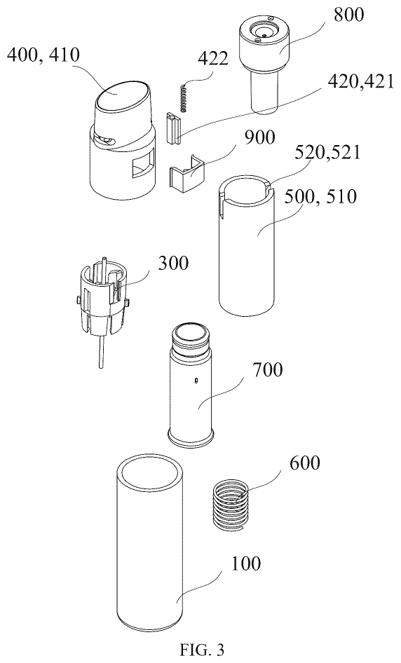

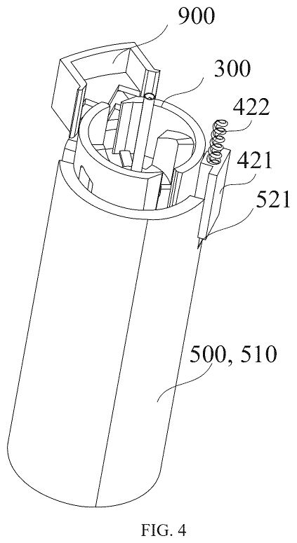

FIG. 1 shows a front view of the atomizer 1000 according to some embodiments of the disclosure; FIG. 2 shows a cross-sectional view of the atomizer 1000 in FIG. 1 taken along R-R; FIG. 3 shows an exploded view of the atomizer 1000 in FIG. 1; and FIG. 4 shows a perspective view of some components of the atomizer 1000 in FIG. 1. As shown in FIGS. I to 4, the atomizer 1000 may include a delivery tube holder 300, a mouthpiece 400 and an actuating member 500. A first end of the mouthpiece 400 is configured to be placed at the mouth of a user, and the mouthpiece 400 includes a mouthpiece body 410 and a first locking structure 420 arranged on the mouthpiece body 410. The actuating member 500 is located at a second end of the mouthpiece 400 opposite to the first end, and includes an actuating member body 510 and a second locking structure 520 arranged on the actuating member body 510. The actuating member 500 can be pivoted relative to the mouthpiece 400 to drive the delivery tube holder 300 to pivot. The actuating member 500 has a locked position and an unlocked position. In the locked position, the first locking structure 420 engages with the second locking structure 520 to block the actuating member 500 from pivoting relative to the mouthpiece 400; and in the unlocked position, the first locking structure 420 does not engage with the second locking structure 520, such that the actuating member 500 can pivot relative to the mouthpiece 400.

In the related art, the housing 100 and/or an actuating member 500 are/is usually pivoted to enable a delivery tube holder 300 to pivot, such that the delivery tube holder 300 pivots downward to the lowest position to enter the pre-triggering state. In this case, if the user continues to pivot the housing 100 and/or the actuating member 500 or causes the atomizer 1000 to slip off, the atomizer 1000 may be automatically triggered, which affects normal drug delivery of the atomizer 1000. Compared with the related art, locking structures are provided on the mouthpiece 400 and the actuating member 500, respectively, to lock or release the actuating member 500 relative to the mouthpiece, such that the actuating member 500 is effectively locked relative to the fixing member (i.e., the mouthpiece) when the atomizer 1000 enters the pre-triggering state, which prevents the automatic triggering of the atomizer 1000.

In some embodiments, as shown in FIGS. 2 and 3, the mouthpiece 400 may be sleeved on the nozzle assembly 800 and/or the delivery tube holder 300, such that an atomized medicinal liquid is sprayed from one end of the mouthpiece 400.

In some embodiments, as shown in FIGS. 2, 3 and 4, the actuating member 500 may have a cylindrical structure, and may be sleeved on the delivery tube holder 300 so as to drive the delivery tube holder 300 to rotate. Alternatively, the actuating member 500 may also be in other shapes, such as an oval shape. In addition, the actuating member 500 may be configured as the housing of the atomizer 1000, or the actuating member 500 is connected to a housing 100 of the atomizer 1000, such that the delivery tube holder 300 can rotate or move in a spiral manner as the housing 100 rotates.

In some embodiments, as shown in FIGS. 3 and 4, the first locking structure 420 may include a locking slider 421, and correspondingly, the second locking structure 520 may include a slot 521 for accommodating the locking slider 421. In this case, in the locked position, the locking slider 421 may be inserted into the slot 521 to block the actuating member 500 from pivoting relative to the mouthpiece 400, as shown in FIG. 4. In the implementation described above, the actuating member 500 can be effectively locked by means of a fixing member (i.e., the mouthpiece), which improves the locking reliability of the actuating member and thus avoids the automatic triggering of the atomizer 1000 as much as possible. In some other embodiments, the first locking structure 420 may include a slot, and correspondingly, the second locking structure 520 may include a locking slider. Alternatively, the first locking structure 420 and the second locking structure 520 may also have other structures that can be selectively engaged and separated, such as a snap-fit structure, to lock or unlock the actuating member 500 relative to the mouthpiece.

In some embodiments, as shown in FIGS. 4, 5B and 6B, the locking slider 421 can move relative to the mouthpiece body 410 in the axial direction L of the mouthpiece 400, and the slot 521 extends in the axial direction L from an end surface of the actuating member body 510 facing the mouthpiece 400. By means of providing the locking slider 421 that can move axially and the slot 521 that extends in the axial direction L as described above, the locking slider 421 can slide into the slot 521 by its own gravity when facing the slot 521, so as to lock the actuating member 500. Alternatively, the locking slider may also move in the radial direction J relative to the mouthpiece body 410, and correspondingly, the slot extends from a circumferential surface of the actuating member body 510 in the radial direction J. The disclosure is not limited to the example described above.

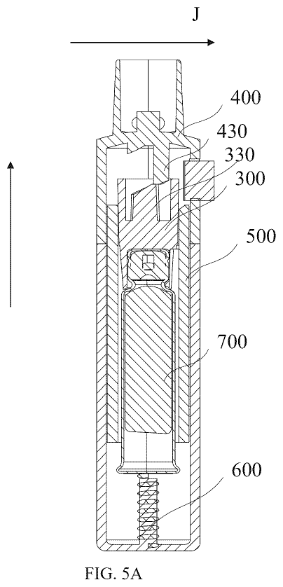

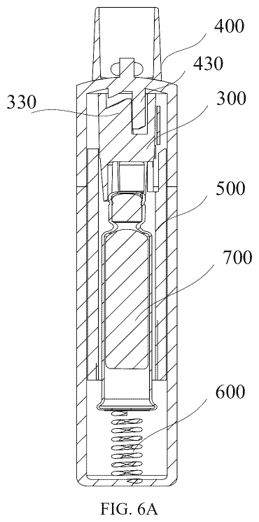

In some embodiments, in the locked position, a portion of the locking slider 421 in the axial direction L is inserted into the slot 521 to block the actuating member 500 from pivoting relative to the mouthpiece 400; and in the unlocked position, the locking slider 421 abuts on the end surface of the actuating member body 510 facing the mouthpiece 400. Specifically, in the unlocked position, the locking slider 421 on the mouthpiece body 410 abuts on the end surface of the actuating member body 510 facing the mouthpiece 400. In this case, during the pivoting of the actuating member 500 relative to the mouthpiece 400, the locking slider 421 may slide on the end surface of the actuating member body 510 facing the mouthpiece 400 until the locking slider 421 is about to directly face the slot 521 in the actuating member body 510, as shown in FIG. 7A. Then, the actuating member 500 continues to be pivoted to allow the locking slider 421 to directly face the slot 521, and at this time, a portion of the locking slider 421 slides into the slot 521 of the actuating member body 510, such that the mouthpiece 400 and the actuating member 500 block each other, as shown in FIG. 5B.

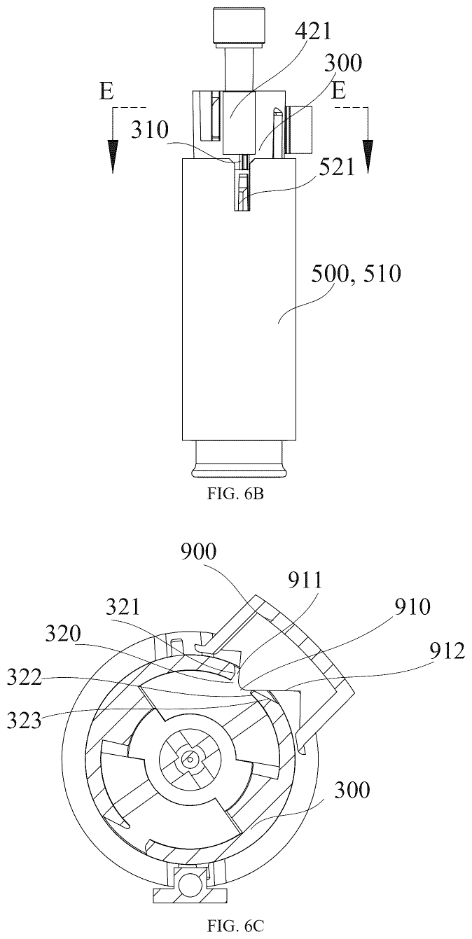

In some embodiments, as shown in FIG. 8, a side wall of the slot 521 may be provided with an inclined surface that is inclined from the side wall toward the end surface of the actuating member body 510 facing the mouthpiece 400. This makes it easy for the locking slider 421 to slide into the slot 521 when facing the slot 521, and also makes it easy for the locking slider 421 to return to and abut on the end surface of the actuating member body 510 facing the mouthpiece 400 after the locking slider 421 returns to the mouthpiece body 410.

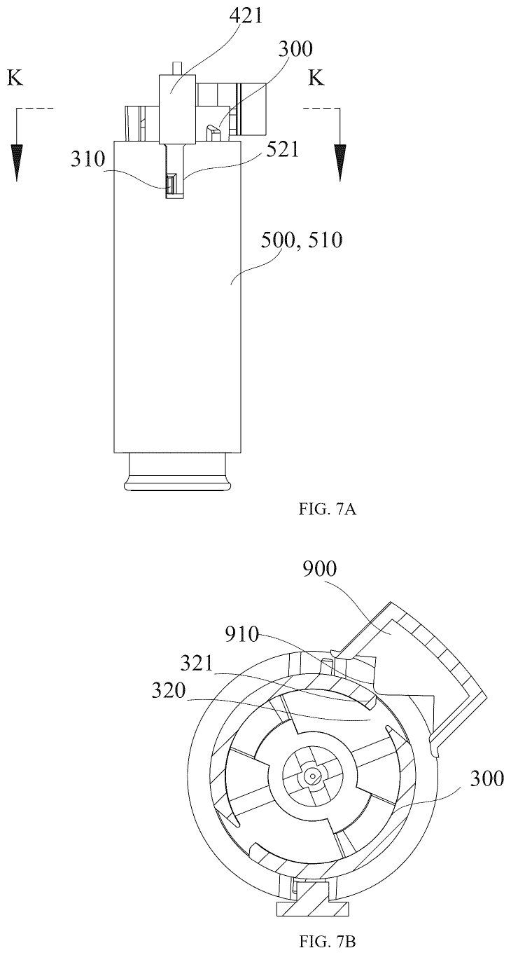

In some embodiments, as shown in FIG. 9, the locking slider 421 may have a T-shaped cross-section. The disclosure is not limited thereto. In addition, a hole 423 may be provided in the locking slider 421 for accommodating an elastic member 422 (described in detail below).

In some embodiments, as shown in FIGS. 3, 4 and 10, the mouthpiece body 410 may include a recess 411 for accommodating the locking slider 421, and the first locking structure 420 further includes the clastic member 422. One end of the clastic member 422 is fixed to an inner wall of the recess 411, and the other end of the clastic member 422 is fixed to the locking slider 421. The locking slider 421 may slide in the recess 411 of the mouthpiece body 410. When the actuating member 500 is in the locked position, a portion of the locking slider 421 may remain in the recess 411 of the mouthpiece body 410, and the other portion of the locking slider is inserted into the slot 521 of the actuating member body 510, such that the mouthpiece 400 and the actuating member 500 block each other. In addition, providing the elastic member may further promote the movement of the locking slider 421. In some examples, the elastic member may be a spring, etc. Alternatively, the first locking structure 420 may include no clastic member. In this case, when the locking slider 421 faces the slot 521, the locking slider 421 may slide into the slot 521 only by its own gravity. In some other embodiments, the first locking structure 420 may further include other drive devices, such as an electric motor, to control the axial movement of the locking slider 421.

In some embodiments, when the actuating member 500 is in the unlocked position, the clastic member 422 is in a compressed state; and when the actuating member 500 is switched from the unlocked position to the locked position, the elastic member 422 returns to promote the locking slider 421 to enter the slot 521. The clastic force of the elastic member 422 may further promote the locking slider 421 to be inserted into the slot 521 quickly and accurately, thereby improving the locking reliability of the actuating member 500.

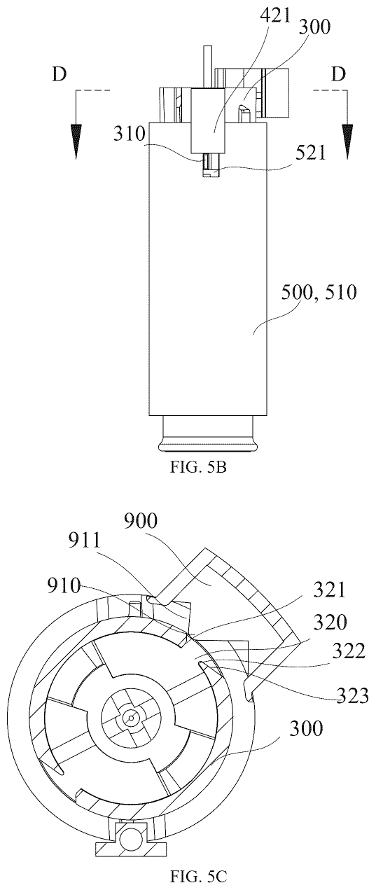

In some embodiments, as shown in FIGS. 5B, 6B and 11, a protrusion 310 is provided on the circumferential surface of the delivery tube holder 300. The protrusion 310 extends into the slot 521. In the locked position, the locking slider 421 abuts on the protrusion 310, such that when the delivery tube holder 300 moves toward the mouthpiece, the protrusion 310 pushes the locking slider 421 to return to the mouthpiece body 410. Specifically, as shown in FIG. 5B, in the locked position, the locking slider 421 is inserted into the slot 521 of the actuating member body 510 and abuts on the protrusion 310 of the delivery tube holder 300, so as to lock the actuating member 500. Then, as shown in FIG. 6B, for example, a switch button 900 can be pressed to enable the delivery tube holder 300 to continue to rotate, such that the delivery tube holder 300 can move toward the mouthpiece 400 under the action of the bottom spring (at this time, the atomizer 1000 is switched from the pre-triggering state to the triggered state) and drive, via the protrusion 310, the locking slider 421 to return into the mouthpiece body. At this time, the actuating member 500 returns to the unlocked position, the actuating member 500 continues to be rotated to enable the locking slider 421 to abut again on the end surface of the actuating member body 510 facing the mouthpiece, and thus the atomizer 1000 returns to the initial state and is ready for the next spraying operation.

In some embodiments, as shown in FIG. 7A, in the unlocked position, the protrusion 310 comes into contact with the slot 521, such that the actuating member 500 can push the delivery tube holder 300 to pivot via the protrusion 310. According to the implementation described above, the protrusion 310 on the delivery tube holder 300 not only enables the locking slider 421 to return to the mouthpiece, but also serves as a transmission member to transmit the rotational motion of the actuating member 500 to the delivery tube holder 300, which simplifies the structure of the atomizer 1000, thereby making the atomizer 1000 more compact and reducing the cost. In some other embodiments, the actuating member may drive the delivery tube holder 300 to rotate by means of a transmission member having other structures, for example, a protrusion provided on the actuating member body 510.

In some embodiments, when the atomizer 1000 is in the initial state, the actuating member 500 is in the unlocked position; and when the atomizer 1000 is in the pre-triggering state, the actuating member 500 is in the locked position. Thus, when the atomizer 1000 is in the initial state, the actuating member 500 is in the unlocked position, and at this time, the actuating member 500 may be pivoted to drive the delivery tube holder 300 to pivot, so as to enable the atomizer 1000 to enter the pre-triggering state. When the atomizer 1000 enters the pre-triggering state, the actuating member 500 is in the locked position, and at this time, the actuating member 500 cannot continue to be rotated, which can prevent the automatic triggering of the atomizer 1000.

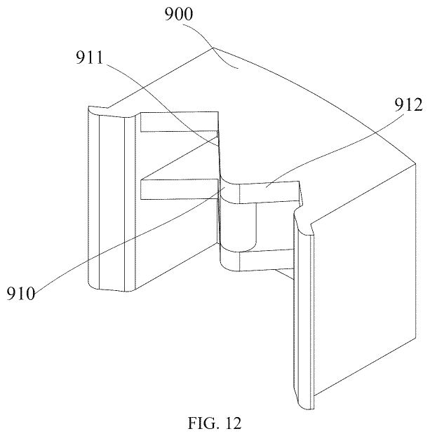

In some embodiments, as shown in FIGS. 4, 5C, 6C, 7B and 12, the atomizer 1000 may further include a switch button 900. The switch button 900 can be pressed in the locked position to drive the delivery tube holder 300 to pivot, so as to enable the atomizer 1000 to enter the triggered state from the pre-triggering state.

In some embodiments, as shown in FIG. 5C, 6C, 7B and 12, a first inclined surface 911 is provided at an end portion 910 of the switch button 900 facing the delivery tube holder 300, and the first inclined surface 911 is configured to be inclined from the end 910 away from the delivery tube holder 300; and an opening 320 is provided in a circumferential wall of the delivery tube holder 300, and the opening 320 is configured to accommodate the end portion 910. Thus, when the switch button 900 is pressed, the first inclined surface 911 on the switch button 900 may push a side wall on the opening 320 of the delivery tube holder 300 so as to push the delivery tube holder 300 to pivot.

In some embodiments, as shown in FIGS. 5C, 6C, 7B and 12, a third inclined surface 912 opposite to the first inclined surface 911 is further provided at the end portion of the switch button 900, and the third inclined surface 912 is configured to be inclined from the end portion 910 away from the delivery tube holder 300, which can reduce resistance to make it easy for a second inclined surface 323 (described in detail below) of the delivery tube holder 300 to come into contact with the end portion 910 of the switch button 900.

In some embodiments, as shown in FIG. 5C, in the locked position, the end portion 910 faces a first side wall 321 of the opening 320, and during the process of the switch button 900 being pressed, the first inclined surface 911 can come into contact with the first side wall 321 of the opening 320 to push the delivery tube holder 300 to pivot. With the arrangement in which the end portion 910 of the switch button 900 faces the first side wall 321 of the opening 320 of the delivery tube holder 300 when in the locked position, when the switch button 900 is pressed, the end portion 910 of the switch button 900 may enter the opening 320 to cause the first inclined surface 911 to come into contact with the first side wall 321 of the opening 320. In this way, the delivery tube holder 300 can be driven to rotate by means of the switch button 900 to enable the atomizer 1000 to enter the triggered state from the pre-triggering state.

In some embodiments, as shown in FIG. 7B, in the unlocked position, the end portion faces the circumferential wall of the delivery tube holder 300. This can ensure that the switch button 900 cannot be pressed when the atomizer 1000 does not enter the pre-triggering state, thereby ensuring the normal drug delivery of the atomizer 1000.

In some embodiments, as shown in FIGS. 5C, 6C, 7B and 12, a second side wall 322 of the opening 320 facing the first side wall 321 is provided with a second inclined surface 323 inclined from the second side wall 322 toward the switch button 900; and as shown in FIG. 6C, when the atomizer 1000 is in the triggered state, the actuating member 500 is pivoted to cause the second inclined surface 323 to come into contact with the end portion 910, so as to enable the switch button 900 to move away from the delivery tube holder 300. Specifically, after the switch button 900 is pressed to enable the atomizer 1000 to enter the triggered state, the actuating member 500 returns to the unlocked position, and at this time, the actuating member 500 may be rotated to drive the delivery tube holder 300 to rotate, so as to cause the second inclined surface 323 of the opening 320 of the delivery tube holder 300 to come into contact with the end portion 910 of the switch button 900. In this way, the rotation of the delivery tube holder 300 can push the switch button 900 to return to the initial state to be ready for the next spraying operation.

To this end, the actuating member 500 can drive the delivery tube holder 300 to rotate in the unlocked position to switch the atomizer 1000 from the initial state to the pre-triggering state, and the switch button 900 can be pressed in the locked position to push the delivery tube holder 300 to continue to rotate, so as to enable the atomizer 1000 to enter the triggered state from the pre-triggering state. After the atomizer 1000 enters the triggered state, the actuating member 500 also returns to the unlocked position. At this time, the actuating member 500 can be rotated to drive the delivery tube holder 300 to rotate, so as to enable the atomizer 1000 to return to the initial state.

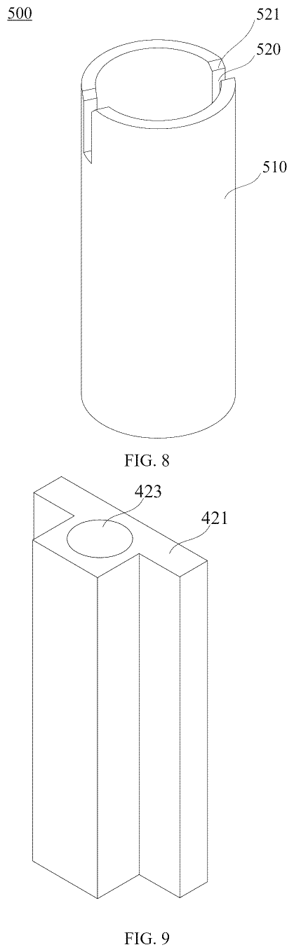

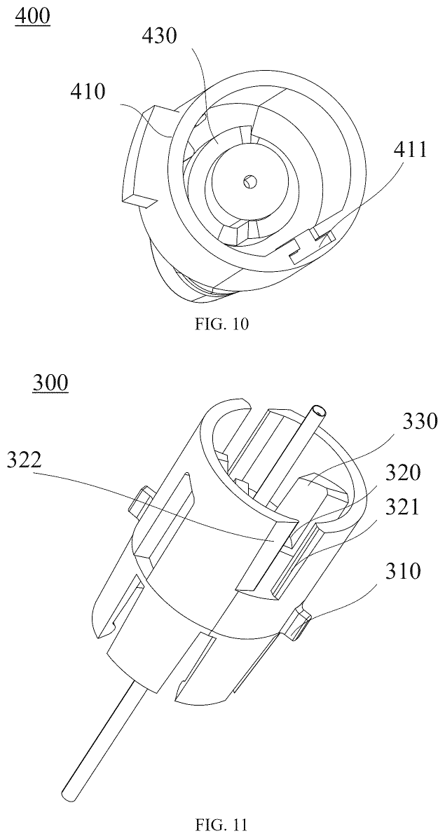

In some embodiments, as shown in FIGS. 10 and 11, a first spiral portion 430 is provided on an inner wall of the mouthpiece 400, and a second spiral portion 330 is provided on an inner wall of the delivery tube holder 300 facing the inner wall of the mouthpiece 400. The first spiral portion 430 can come into contact with the second spiral portion 330, so as to enable the delivery tube holder 300 to move in the axial direction L while pivoting. The first spiral portion 430 protrudes from an inner bottom wall of the mouthpiece body 410 toward the delivery tube holder 300, and the second spiral portion 330 may protrude radially inward from an inner wall of a tube holder body of the delivery tube holder 300. The first spiral portion 430 may cooperate with the second spiral portion 330. Thus, during the rotation of the delivery tube holder 300, the delivery tube holder 300 may move in the axial direction L. In the disclosure, during the downward movement of the delivery tube holder 300, the delivery tube holder 300 moves downward while rotating under the action of the first spiral portion and the second spiral portion, that is, performs a composite motion of rotation and downward translation; and during the upward movement of the delivery tube holder 300, the delivery tube holder 300 can perform a simple upward translation movement without any rotation.

In some embodiments, in the pre-triggering state, the highest point of the first spiral portion 430 in the axial direction L coincides with the highest point of the second spiral portion 330 in the axial direction L. Thus, in the pre-triggering state, the delivery tube holder 300 is at the lowest point in the axial direction L, such that the bottom spring 600 on the bottom of the delivery tube holder 300 can be compressed to the maximum extent; and in the triggered state, after the first spiral portion 430 is separated from the second spiral portion 330, the delivery tube holder 300 moves toward the mouthpiece 400 under the upward thrust of the bottom spring 600, so as to enable the nozzle assembly 800 to atomize and spray the medicinal liquid in the container 700 connected to the delivery tube holder 300.

In some embodiments, the width of the first spiral portion 430 in the radial direction J of the delivery tube holder 300 is different from the width of the second spiral portion 330 in the radial direction J. This can reduce the frictional force when the first spiral portion 430 and the second spiral portion 330 slide relatively, such that the user's operation is more labor-saving.

In some embodiments, the atomizer 1000 may further include other members, such as a housing 100, a container 700 for holding the liquid to be atomized, a bottom spring 600 for applying a thrust to the delivery tube holder 300, a spraying assembly 800 for atomizing the liquid and spraying the atomized liquid, and a dust cover 200.

According to some embodiments of the disclosure described above, the atomizer 1000 may be, but does not necessarily, operated as follows.

When the atomizer 1000 is in the initial state, the first spiral portion 430 on the inner wall of the mouthpiece 400 is separated from the second spiral portion 330 on the inner wall of the delivery tube holder 300, the locking slider 421 on the mouthpiece 400 abuts on the end surface of the actuating member body 510 facing the mouthpiece 400, and the end portion 910 of the switch button 900 faces the circumferential wall of the delivery tube holder 300.

From the initial state, the user holds the housing 100 and rotates the housing 100 by an angle, and the actuating member 500 drives the delivery tube holder 300 to rotate along with the housing 100, such that the first spiral portion 430 on the mouthpiece 400 comes into contact with the second spiral portion 330 on the delivery tube holder 300. At this time, the delivery tube holder 300 may move downward in a spiral manner under the action of the first spiral portion 430 and the second spiral portion 330. As the actuating member 500 rotates, the slot 521 in the actuating member 500 approaches the locking slider 421 on the mouthpiece 400, as shown in FIG. 7A, and the end portion 910 of the switch button 900 also approaches the first side wall 321 of the opening 320 of the delivery tube holder 300, as shown in FIG. 7B. The actuating member 500 continues to rotate until the highest point of the first spiral portion 430 comes into contact with the highest point of the second spiral portion 330, and at this time, the atomizer 1000 enters the pre-triggering state, as shown in FIG. 5A.

When the atomizer 1000 is in the pre-triggering state, the bottom spring 600 of the delivery tube holder 300 is compressed to the maximum extent, as shown in FIG. 5A. Also, a portion of the locking slider 421 on the mouthpiece 400 is inserted into the slot 521 of the actuating member body 510 and abuts on the protrusion 310 on the circumferential wall of the delivery tube holder 300, such that the actuating member 500 is in the locked position, as shown in FIG. 5B. In addition, the end portion 910 of the switch button 900 faces the first side wall 321 of the delivery tube holder 300, as shown in FIG. 5C. In the locked position, if the user does not apply an external force by means of other components, the atomizer 1000 does not perform an atomization or spraying operation, even if the atomizer 1000 is subjected to an external force, such as impact or shaking, or even accidents such as slipping from a height. Thus, the atomizer 1000 is effectively locked in this state.

When the user wants to use the atomizer 1000, the user may apply a radial inward thrust to the switch button 900, that is, press the switch button 900. The switch button 900 is pressed such that the first inclined surface 911 on the switch button 900 comes into contact with the first side wall 321 of the opening 320 of the delivery tube holder 300 and pushes the first side wall 321 to move, so as to drive the delivery tube holder 300 to further rotate. At this time, the protrusion 310 on the delivery tube holder 300 also moves a distance in the slot 521 along with the delivery tube holder 300. In addition, the first spiral portion 430 of the mouthpiece 400 is separated from the second spiral portion 330 of the delivery tube holder 300, and the delivery tube holder 300 moves toward the mouthpiece 400 in the axial direction L under the upward thrust of the bottom spring 600, so as to enable the atomizer 1000 to enter the triggered state, as shown in FIG. 6A. In addition, the protrusion 310 on the delivery tube holder 300 pushes the locking slider 421 to return into the mouthpiece body, so as to enable the actuating member 500 to return to the unlocked position, as shown in FIG. 6B.

Then, the user continues to rotate the housing 100 by an angle to enable the actuating member 500 to continue to drive the delivery tube holder 300 to rotate along with the housing 100. At this time, the locking slider 421 returns to and abuts on the end surface of the actuating member body 510 facing the mouthpiece 400. In addition, as shown in FIG. 6C, the second inclined surface 323 of the second side wall 322 of the delivery tube holder 300 comes into contact with the end portion 910 of the switch button 900 and pushes the switch button 900 to return to the original position, such that the atomizer 1000 returns to the initial position. At this point, the atomizer 1000 completes one stroke cycle of spaying.

In some embodiments, two first spiral portions 430, two second spiral portions 330, two openings 320, two slots 521, etc. may be symmetrically arranged in the corresponding components of the atomizer 1000, such that the atomizer 1000 can perform two spraying operations when rotating one cycle, that is, having two stroke cycles.

What is claimed is:

1. An atomizer, comprising:
a delivery tube holder;
a mouthpiece, which has a first end configured to be placed at a mouth of a user, and comprises a mouthpiece body and a first locking structure arranged on the mouthpiece body; and
an actuating member, which is located at a second end of the mouthpiece opposite to the first end, and comprises an actuating member body and a second locking structure arranged on the actuating member body,
wherein the actuating member is capable of being pivoted relative to the mouthpiece to drive the delivery tube holder to pivot; and
wherein the actuating member has a locked position, in which the first locking structure engages with the second locking structure to block the actuating member from pivoting relative to the mouthpiece, and an unlocked position, in which the first locking structure does not engage with the second locking structure such that the actuating member is capable of pivoting relative to the mouthpiece.

2. The atomizer according to claim 1, wherein the first locking structure comprises a locking slider, and the second locking structure comprises a slot for accommodating the locking slider.

3. The atomizer according to claim 2, wherein the locking slider is capable of moving relative to the mouthpiece body in an axial direction of the mouthpiece, and the slot extends in the axial direction from an end surface of the actuating member body facing the mouthpiece.

4. The atomizer according to claim 3, wherein
in the locked position, a portion of the locking slider in the axial direction is inserted into the slot to block the actuating member from pivoting relative to the mouthpiece; and
in the unlocked position, the locking slider abuts on the end surface of the actuating member body facing the mouthpiece.

5. The atomizer according to claim 2, wherein the mouthpiece body comprises a recess for accommodating the locking slider, and the first locking structure further comprises an elastic member, one end of the elastic member being fixed to an inner wall of the recess, and the other end of the elastic member being fixed to the locking slider.

6. The atomizer according to claim 5, wherein
in the unlocked position, the elastic member is in a compressed state; and
during the movement of the actuating member from the unlocked position to the locked position, the elastic member returns to enable the locking slider to enter the slot.

7. The atomizer according to claim 4, wherein a circumferential surface of the delivery tube holder is provided with a protrusion extending into the slot, and in the locked position, the locking slider abuts on the protrusion, such that during the movement of the delivery tube holder toward the mouthpiece, the protrusion pushes the locking slider to return to the mouthpiece body.

8. The atomizer according to claim 7, wherein in the unlocked position, the protrusion comes into contact with the slot, such that the actuating member is capable of pushing the delivery tube holder to pivot via the protrusion.

9. The atomizer according to claim 4, wherein a side wall of the slot is provided with an inclined surface inclined from the side wall toward the end surface.

10. The atomizer according to claim 1, wherein
when the atomizer is in an initial state, the actuating member is in the unlocked position; and
when the atomizer is in a pre-triggering state, the actuating member is in the locked position.

11. The atomizer according to claim 1, further comprising a switch button, wherein in the locked position, the switch button is capable of being pressed to drive the delivery tube holder to pivot.

12. The atomizer according to claim 11, wherein
a first inclined surface is provided at an end portion of the switch button facing the delivery tube holder, and the first inclined surface is configured to be inclined from the end portion away from the delivery tube holder; and
an opening is provided in a circumferential wall of the delivery tube holder, and the opening is configured to accommodate the end portion.

13. The atomizer according to claim 12, wherein in the locked position, the end portion faces a first side wall of the opening, and during a process of the switch button being pressed, the first inclined surface is capable of coming into contact with the first side wall to push the delivery tube holder to pivot.

14. The atomizer according to claim 13, wherein in the unlocked position, the end portion faces the circumferential wall of the delivery tube holder.

15. The atomizer according to claim 14, wherein
a second side wall of the opening opposite to the first side wall is provided with a second inclined surface inclined from the second side wall toward the switch button; and
when the atomizer is in a triggered state, the actuating member is pivoted to cause the second inclined surface to come into contact with the end portion, so as to enable the switch button to move away from the delivery tube holder.

16. The atomizer according to claim 12, wherein the end portion of the switch button is further provided with a third inclined surface opposite to the first inclined surface, and the third inclined surface is configured to be inclined from the end portion away from the delivery tube holder.

17. The atomizer according to claim 9,
wherein a first spiral portion is provided on an inner wall of the mouthpiece, and a second spiral portion is provided on an inner wall of the delivery tube holder facing the inner wall of the mouthpiece; and
wherein the first spiral portion is capable of coming into contact with the second spiral portion, so as to enable the delivery tube holder to move in the axial direction while pivoting.

18. The atomizer according to claim 17, wherein in the pre-triggering state, the highest point of the first spiral portion in the axial direction coincides with the highest point of the second spiral portion in the axial direction.

19. The atomizer according to claim 17, wherein a width of the first spiral portion in a radial direction of the delivery tube holder is different from a width of the second spiral portion in the radial direction.
